(19) United States
(12) Patent Application Publication
(10) Pub. No.: US 2004/0116875 A1
(43) Pub. Date: Jun. 17, 2004

(54) Ergonomic syringe barrel

(76) Inventors: Dan E. Fischer, Sandy, UT (US); David Fischer, Colton, CA (US); Bruce S. McLean, Sandy, UT (US); Dan Loveridge, Sandy, UT (US)

Correspondence Address: WORKMAN NYDEGGER (F/K/A WORKMAN NYDEGGER & SEELEY), 60 EAST SOUTH TEMPLE, 1000 EAGLE GATE TOWER, SALT LAKE CITY, UT, 84111, US

(21) Appl. No.: 10/321,258

(22) Filed: Dec. 17, 2002

(51) Int. Cl. A61M 5/315

(52) U.S. Cl. 604/227

(57) ABSTRACT

The syringes of the invention are configured with ergonomic features that include polygonal shaped flanges and textured barrels. The polygonal shaped flanges, which preferably have between five and ten sides, help reduce the ability of the syringes to undesirably roll when they are put down and reduce the material required to manufacture the flanges, yet are more comfortable than square and rectangular flanges. The textured barrels enable a user to grab and hold onto the barrels during delivery with syringes so as to increase the leverage and control over the syringe.

### BACKGROUND OF THE INVENTION

1. The Field of the Invention

The present invention is in the field of fluid delivery devices and, more particularly, to syringes.

2. The Relevant Technology

In the dental industry, syringes are often used in the application of fluid materials such as, but not limited to dental filling materials, sealers, medicines, and local anesthetics. Syringes are particularly useful delivery devices because they can be configured with predetermined dosages and they can also be delivered in a controlled manner. The rate and manner in which the material is expelled from the syringe can typically be controlled by the rate and manner in which a plunger is forced through the syringe.

Existing syringes, such as the syringe 10 illustrated in FIG. 1, typically include a hollow barrel 12 with two ends. At one end, the barrel tapers to a tip 14 or is attached to a tip that is configured to expel the material. The opposite end of the barrel 12 is typically configured with a flange 16 that can be used for gripping when the material is expelled out from the barrel 12. As shown in FIG. 1, the fingers of the hand can bear against the flange 16 of the barrel while the thumb is used to push the plunger 18 through the barrel 12. As the plunger 18 is forced through the barrel 12, the material contained within the barrel 12 is forced out of the barrel 12.

One problem experienced by existing syringes, however, is that the flange is either round or square. Round flanges (not shown) are typically more comfortable on a user's fingers than square flanges and are therefore desirable. However, a round shaped flange typically requires more material to manufacture than a square shaped flange of the same size. Accordingly, syringes having round flanges are typically more expensive to manufacture than syringes having square flanges. Additionally, the round flanges may enable the syringe to roll off of a surface that the syringe is set down upon. This can be a problem because the syringe may undesirably fall onto the floor where it can become contaminated. This can also be somewhat hazardous, such as when the syringe is equipped with a needle that may undesirably pierce the skin of the patient or the practitioner.

Another problem with existing syringes is that the external surface of the barrel 12 is smooth, making it difficult at times, to grip with the fingers. For instance, as shown in FIG. 2, a practitioner may grip the barrel 12 between the fingers with the plunger 18 positioned within the palm of the hand, thereby enabling the practitioner to apply additional leverage to the plunger 18 and to increase the control the practitioner has over the orientation of the syringe 10. This technique is particularly useful when the material within the syringe 10 is somewhat viscous, making it difficult to drive the plunger 18 entirely through the barrel 12 with the thumb. However, slippage can be a problem when using this technique, particularly when the fingers or syringe 10 become wet from water, blood, saliva or other materials that are present in a typical dental and medical environment.

### SUMMARY OF THE INVENTION

Briefly summarized, the present invention is directed to improved syringes having ergonomically improved barrels.

According to one aspect of the invention, the ergonomically improved barrels may include a textured outer surface configured to facilitate gripping and handling of the syringe barrel. The texturing on the barrel may be disposed along the entire outer surface of the barrel or along only a portion of the barrel.

The desired texturing may be provided with a ribbed surface, with a knurled surface, or any other desired texturing. The texturing may be recessed and formed within the barrel surface or protrude out away from the barrel surface. According to one embodiment, the texturing may be integrally formed as a part of the barrel, such as during injection molding of the barrel, in a two-part molding process, or in a machining process. An independent textured surface may also be affixed to the barrel with a chemical or mechanical bond, such as with an adhesive, with a mechanical friction fit, by welding, or any other suitable process.

According to another aspect of the invention, the barrel may be configured with a flange formed in the shape of a polygon having more than four sides. According to one embodiment, the flange has five to ten sides. The polygon shape of the flange minimizes the material that must be used to manufacture the flange, relative to round flanges of the same size. The multiple sides also help to prevent the syringe from rolling when the syringe is set down.

These and other benefits, advantages and features of the present invention will become more fully apparent from the following description and appended claims, or may be learned by the practice of the invention as set forth hereinafter.

### BRIEF DESCRIPTION OF THE DRAWINGS

In order that the manner in which the above recited and other benefits, advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 illustrates a prior art syringe having a smooth exterior barrel surface and a square flange that is disposed in a hand;

FIG. 2 illustrates the prior art syringe of FIG. 1 in which the barrel of the syringe is disposed between two fingers of the hand holding the syringe;

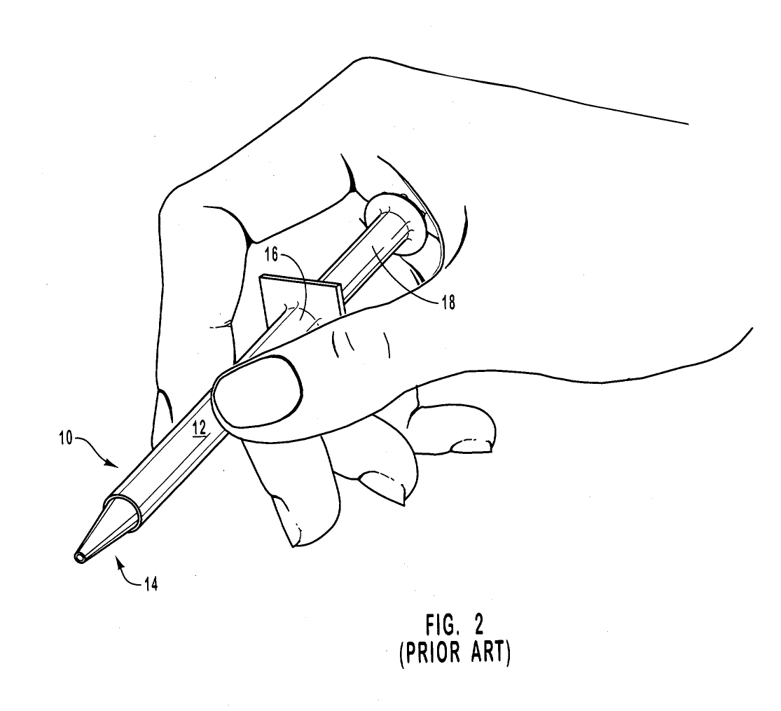

FIG. 3 illustrates one embodiment of the syringe of the invention that includes a barrel having a textured surface covering only a portion of the barrel and an eight-sided polygonal flange;

FIG. 4 illustrates one embodiment of the syringe of the invention that includes a barrel having a textured surface extending almost the entire length of the barrel and a six-sided polygonal flange;

FIG. 5 illustrates one embodiment of the syringe of the invention that includes a barrel having a textured surface that includes rings protruding out away from the barrel and a five-sided polygonal flange;

FIG. 6 illustrates one embodiment of the syringe of FIG. 3 that is held within the hand of a user with some of the user's fingers disposed on the flange; and

FIG. 7 illustrates one embodiment of the syringe of FIG. 3 that is held within the hand of a user with some of the user's fingers disposed on the textured surface of the barrel.

### DETAILED DESCRIPTION OF THE PREFERRED EMBODIMENTS

A detailed description of the invention will now be provided with specific reference to figures illustrating preferred embodiments of the invention. It will be appreciated that like structures will be provided with like reference designations. To provide context for interpreting the scope of the invention, certain terms used throughout the application will now be defined.

The term “fluid material,” as used herein, refers to any composition, fluid, compound, or other material that is capable of being dispensed from a syringe. The term “fluid material is sometimes used interchangeably herein with the terms “material,” and “fluid.”

As mentioned above, the syringes of the invention are configured with ergonomic features that can improve the control a user has in dispensing fluids from the syringes. The ergonomic features, which are described herein, generally include a textured barrel and a polygon shaped flange.

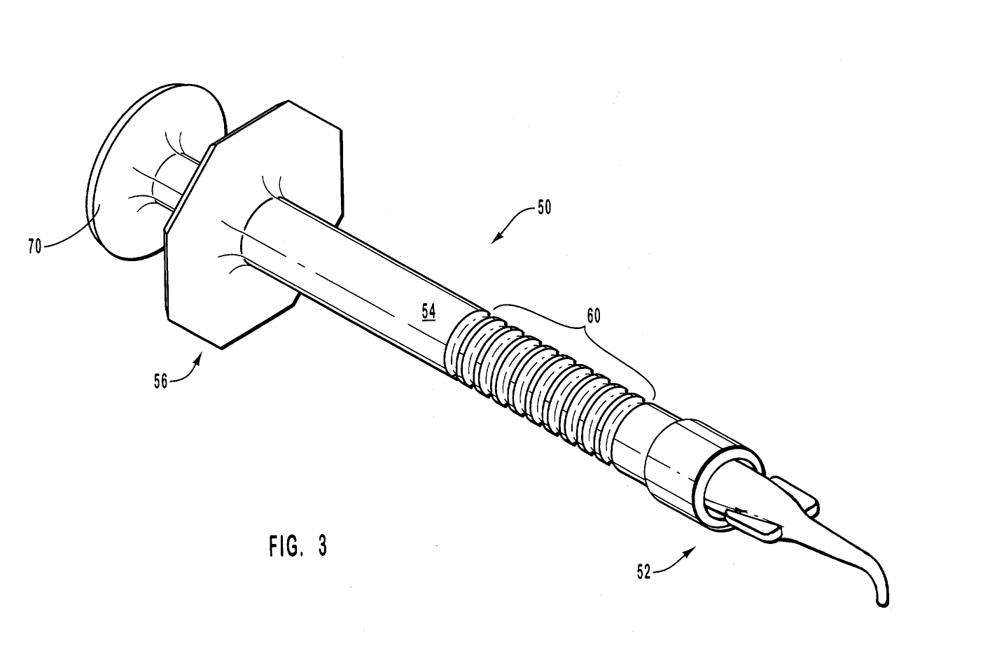

FIG. 3 illustrates one embodiment of the syringe 50 of the invention. As shown, the syringe 50 includes a barrel 54 that is hollow and configured to contain a fluid material. The barrel 50 extends from a first end comprising a tip 52 to a second end comprising a flange 56. The tip 52 is configured to disperse the fluid material and the flange 56 is configured to accommodate the fingers of a user holding the syringe 50.

One unique feature of the flange 56 is that it is polygon shaped. In the present embodiment, for example, the flange 56 is configured with eight sides. The polygon shape of the flange 56 is useful for reducing the amount of material that is required to manufacture the flange 56, relative to similarly sized round flanges, thereby reducing the overall cost of manufacturing the barrel 54 and the syringe 50. The polygonal shape of the flange 56 is also useful because it can help to prevent undesired rolling of the syringe 50 when the syringe 50 is set down.

Some existing syringes include square and rectangular flanges that can help to prevent rolling of the syringe. However, square and rectangular flanges are configured to only be gripped from a few orientations. The sharp 90° angle on existing square and rectangular flanges can also make it uncomfortable to hold and operate the syringe. Accordingly, it will be appreciated that the polygonal shape of the flange 56 disclosed in the present invention is useful for increasing the ergonomic comfort of operation of the syringe 50, particularly in comparison to existing syringes having square and rectangular flanges.

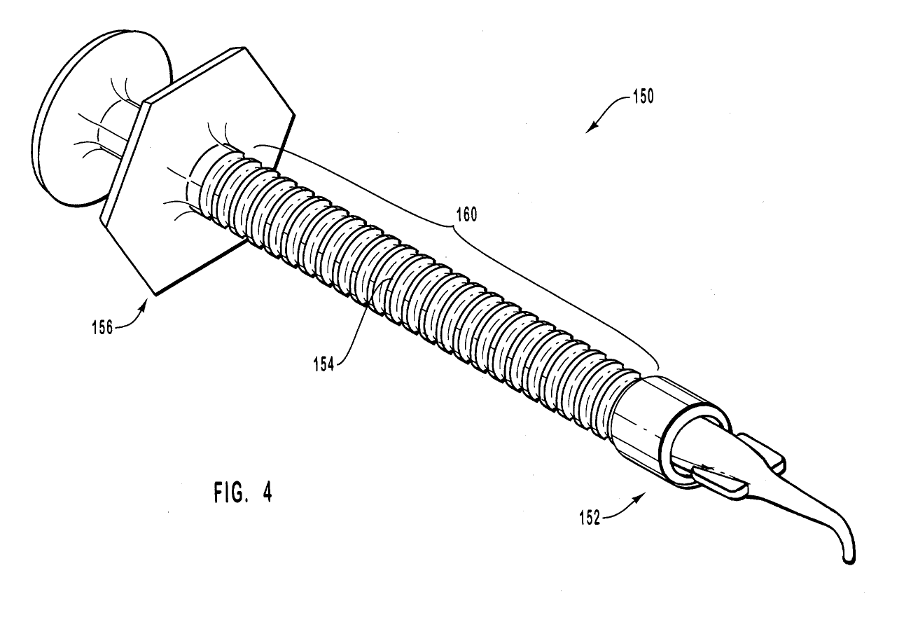

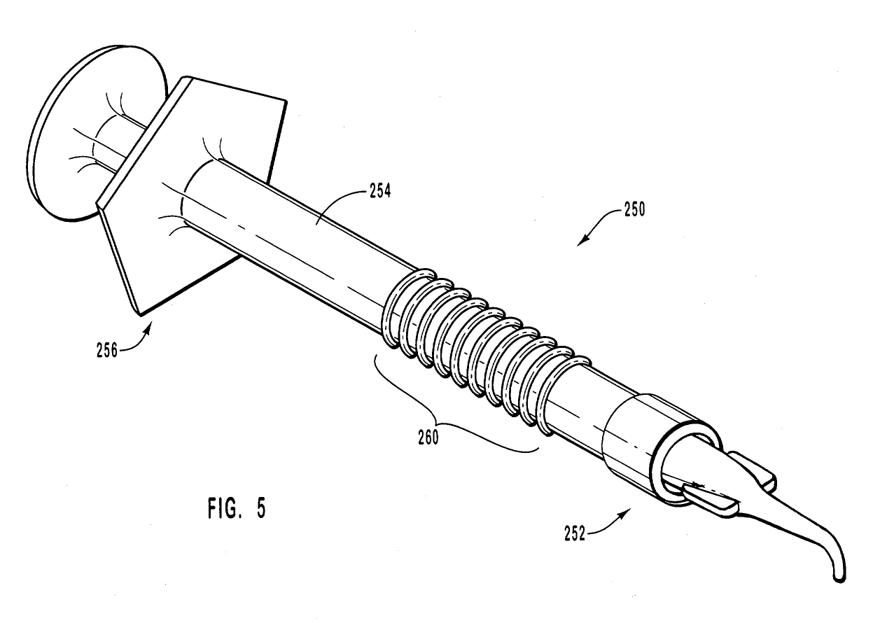

The number of sides that the polygonal flange 56 may be configured with can vary between different embodiments. For instance, the flange 56 illustrated in FIG. 3 includes eight sides, the flange 156 shown in FIG. 4, however, includes only six sides, and the flange 256 shown in FIG. 5 includes only five sides. Preferably, the number of sides on the polygonal flange is greater than four and less than eleven. However, it will be appreciated that the invention is not limited to syringes having flanges of between five and ten sides. In particular, the flanges may be configured with more than ten sides.

FIG. 3 illustrates another aspect of the invention. As shown, the syringe 50 of the invention also includes an ergonomically configured barrel 54. The barrel 54 is considered ergonomically configured because it is textured, making it relatively easier to grip. The textured surface 60 on the barrel 54 can be disposed over only a portion of the barrel 54, as shown, or the textured surface can alternatively be disposed over the entire barrel 54.

FIG. 4 illustrates one embodiment in which the syringe barrel 154 is configured with a textured surface 160 that extends over almost the entire barrel 154. This embodiment is useful for facilitating the gripping of the syringe 150 at any point on the barrel 154. The textured surfaces 60 and 160 of FIGS. 3 and 4 are shown to comprise rings that are formed into the barrels 54 and 154 of the respective syringes 50 and 150. It will be appreciated that by forming the rings into the barrels 54, 154, as illustrated, less material is required to manufacture the barrels 54 and 154 than would otherwise be required to manufacture the barrels 54 and 154. In particular, a void of material exists between each of the rings comprising the textured surfaces 60 and 160, thereby reducing the total material required to manufacture the barrels 54 and 154.

FIG. 5 illustrates another embodiment in which the textured surface 260 is formed by rings that are disposed on top of the barrel 254, rather than being recessed within the barrel 254. Although this barrel 254 may require more material for manufacturing than the barrels 54 and 154 described above, this embodiment is useful because the extra material can provide the barrel 254 with increased strength and stability. Accordingly, the manner in which the textured surfaces 60, 160, 260 are formed may vary to accommodate different needs and preferences. Likewise, the type of texturing used to create the textured surfaces 60, 160, and 260 may also vary. For instance, in other embodiments, the textured surfaces may include knurling, rings, bumps, protrusions, recesses, grooves, or other texturing structures configured to make the barrel ergonomically easier to grip.

According to one embodiment, the textured surfaces are integrally formed with the barrels during injection molding of the barrels, such as during a single-part or a two-part molding process. The textured surfaces may also be formed separately from the barrels and subsequently attached to the barrels, such as with an adhesive, by welding, with a friction fit or a mechanical coupling.

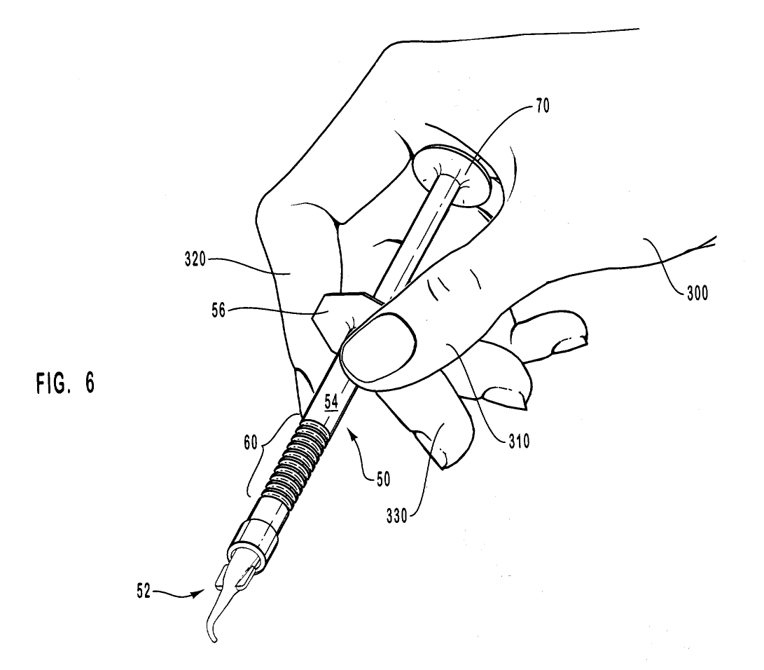

Attention is now directed to FIG. 6 to further illustrate and describe the utility of syringes of the invention. As shown, the inventive syringe 50 is disposed within the hand 300 of a user. The illustrated syringe 50 is the same as the syringe 50 illustrated in FIG. 3, including a polygon shaped flange 56 and a textured surface 60 that is disposed on the barrel 54.

Because the flange 56 has a plurality of sides, as described above, the user can easily grab and hold onto the syringe 50 by the flange 56 from a variety of different orientations. For instance, the flange 56 may be held by the thumb 310, the index finger 320, and the pointer finger 330, or with any other combination of fingers. When the syringe 50 is held in the illustrated orientation, then the plunger 70 is able to rest against the palm of the hand 300.

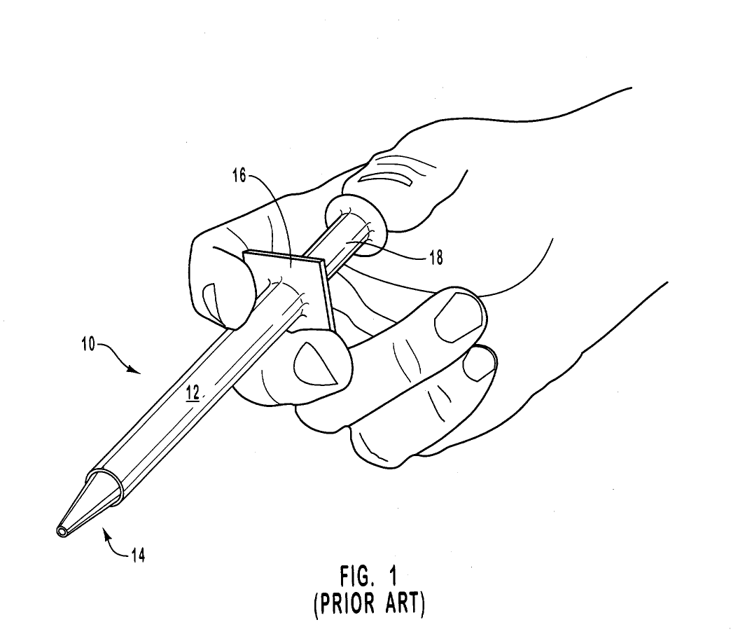

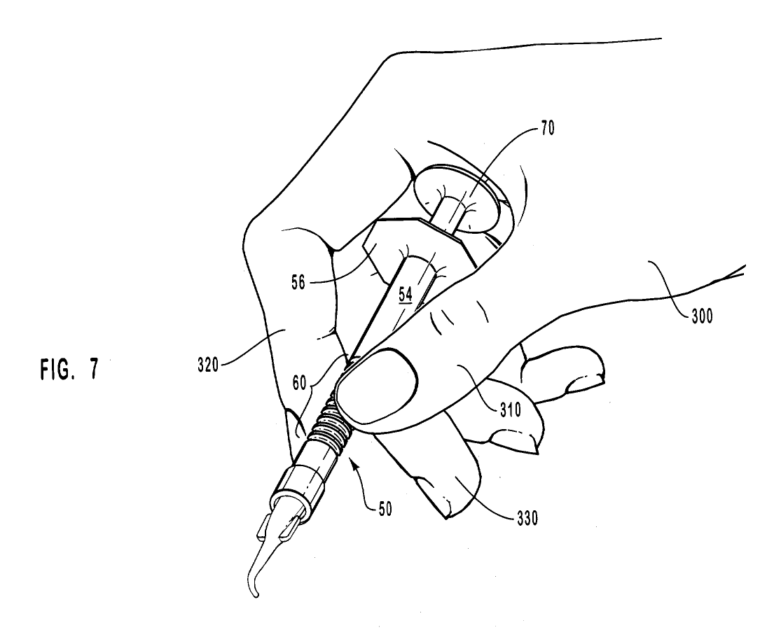

From this position, the user can pull on the flange 56 with the fingers, causing the palm of the hand to force the plunger 70 through the barrel 50, thereby expelling the fluid that is contained within the barrel 50 out of the barrel 50 through the tip 52. However, once the plunger 70 is forced a certain distance into the barrel 54, it can become difficult to continue applying an adequate pulling force to the flange 56 with the fingers at the same time an adequate pushing force is applied to the plunger 70 with the palm of the hand. It may also be difficult to express the fluid material out of the barrel 54 with the desired control when the thumb 310 is used to apply the pushing force to the plunger, as generally illustrated in FIG. 1, when the other fingers are disposed on the flange 56. To exercise desired control, therefore, it may be desirable to move the fingers up about the barrel 54 to obtain a more convenient and ergonomic hold on the syringe 54, as illustrated in FIG. 7.

FIG. 7 illustrates one embodiment in which the fingers 310, 320 and 330 have been moved up to grasp a hold of the syringe 50 by the barrel 54. As mentioned above, it may be desirable to hold the syringe 50 in this manner to provide a sufficient grip for expelling the residual amounts of the fluid material out of the syringe 50.

As shown, the user can now easily pull on the barrel 54 of the syringe 50 with the fingers 310, 320 and 330, while at the same time pushing on the plunger 70 with the palm of the hand 300. It will be appreciated that the textured surface 60 is particularly useful in this embodiment for providing increased friction for enabling the fingers to more easily grab and hold onto the barrel 54 than would otherwise be possible, particularly when the barrel 54 is wet.

In summary, the syringes of the invention provide ergonomic advantages over existing syringes in the art. In particular, the syringes of the present invention include polygonal shaped flanges and textured barrel surfaces to increase the operational comfort and control of the syringes. In certain embodiments, the polygonal shaped flanges and textured surfaces also reduce the amount of material required to manufacture the syringes. It should be appreciated that these are improvements over the existing devices in the art.

It will also be appreciated that the present claimed invention may be embodied in other specific forms without departing from its spirit or essential characteristics. The described embodiments are to be considered in all respects only as illustrative, not restrictive. The scope of the invention is, therefore, indicated by the appended claims rather than by the foregoing description. All changes that come within the meaning and range of equivalency of the claims are to be embraced within their scope.

What is claimed is:

1. A syringe configured to contain and deliver a fluid material, the syringe comprising:
a hollow barrel configured to contain a fluid material therein, the barrel extending from a first end to a second end; and
a textured surface disposed over at least a portion of the barrel, the textured surface being configured to be engaged by the fingers of a person holding the syringe.

2. A syringe as recited in claim 1, further comprising a plunger that is configured to be inserted within the first end of the barrel and to express the fluid material out of the second end of the barrel when the plunger is advanced through the barrel.

3. A syringe as recited in claim 1, further comprising a polygonal-shaped flange that protrudes laterally away from an outer surface of the barrel.

4. A syringe as recited in claim 3, the flange comprising the shape of a polygon having at least five sides.

5. A syringe as recited in claim 3, the flange comprising the shape of a polygon having ten or fewer sides.

6. A syringe as recited in claim 5, the flange comprising the shape of a polygon having eight sides.

7. A syringe as recited in claim 1, the textured surface being integrally attached to the barrel.

8. A syringe as recited in claim 1, the textured surface protruding away from the barrel.

9. A syringe as recited in claim 1, the textured surface comprising rings formed around the barrel.

10. A syringe as recited in claim 1, the textured surface being disposed over a portion of the barrel.

11. A syringe as recited in claim 1, the textured surface comprising at least one of a knurled surface, recesses, grooves, ridges, or bumps.

12. A syringe configured to contain and deliver a fluid material, the syringe comprising:
a hollow barrel configured to contain a fluid material therein, the barrel extending from a first end to a second end; and
a polygonal shaped flange having at least five sides, the polygonal shaped flange protruding laterally away from an outer surface of the barrel and being configured to be engaged by the fingers of a person holding the syringe.

13. A syringe as recited in claim 12, the flange comprising the shape of a polygon having less than eleven sides.

14. A syringe as recited in claim 12, further comprising a textured surface disposed over at least a portion of the barrel, the textured surface being configured to be engaged by the fingers of a person holding the syringe.

15. A syringe as recited in claim 12, the textured surface comprising at least one of rings, a knurled surface, recesses, grooves, ridges, or bumps.

16. A syringe as recited in claim 12, further comprising a plunger that is at least partially disposed within the barrel and that is configured to express the fluid material out of the barrel when the plunger is advanced through the barrel.

17. A syringe configured to contain and deliver a fluid material, the syringe comprising:
a hollow barrel configured to contain a fluid material, the barrel extending from a first end to a second end;
a polygonal shaped flange having at least five sides that protrudes laterally away from an outer surface of the barrel and that is configured to be engaged by the fingers of a person holding the syringe;
a textured surface disposed over at least a portion of the barrel, the textured surface being configured to be grasped by the fingers of a person holding the syringe; and
a plunger configured to be inserted within the first end of the barrel and to express the fluid material out of the second end of the barrel when the plunger is advanced through the barrel.

18. A syringe as recited in claim 17, the flange comprising the shape of a polygon having ten or fewer sides.

19. A syringe as recited in claim 17, the textured surface comprising at least one of rings, a knurled surface, recesses, grooves, ridges, or bumps.

20. A syringe as recited in claim 17, the textured surface being integrally attached to the barrel.
